(19) United States
(12) Patent Application Publication
(10) Pub. No.: US 2025/0339239 A1
(43) Pub. Date: Nov. 6, 2025

(54) DYNAMIC PRESCRIPTION INTERFACES

(71) Applicant: Align Technology, Inc., San Jose, CA (US)

(72) Inventors: Jacob William BOWLAND, Flower Mound, TX (US); Lavanya Naganath IYER, Ontario (CA); Fangjia TIAN, San Jose, CA (US); Rohit TANUGULA, San Jose, CA (US); Bastien PESENTI, San Jose, CA (US); Surbhi GUPTA, Fremont, CA (US); Michael Austin BROWN, Orlando, FL (US); Leon RASOVSKY, Mountain View, CA (US); Brent BELLINGER, Washington, UT (US); Somesh PERI, Union City, CA (US); Savva KUZMIN, Ontario (CA); Srinivas KAZA, Mountain View, CA (US); Thomas JANSEN, Durham, NC (US)

(21) Appl. No.: 19/196,689

(22) Filed: May 1, 2025

Related U.S. Application Data
(60) Provisional application No. 63/641,408, filed on May 1, 2024.

(51) Int. Cl. A61C 7/00; A61C 9/00; G05B 19/04

(52) U.S. Cl. CPC A61C 7/002; A61C 9/004; G05B 19/0405

(57) ABSTRACT

Dynamic prescription interfaces, including systems and apparatuses assisting a dental professional (e.g., dentist) in prescribing one or more dental appliances (e.g., retainers, expanders, etc.). These methods and apparatuses may permit the dynamic display of the dental appliance as the dental professional modifies the prescription, in real-or near-real time.

### CLAIM OF PRIORITY

This patent application claims priority to U.S. provisional patent application No. 63/641,408, titled “DYNAMIC PRESCRIPTION INTERFACES,” filed on May 1, 2024 and herein incorporated by reference in its entirety.

### BACKGROUND

A typical dental or orthodontic prescription may be completed by a dental professional either manually or electronically by providing instructions to multiple questions sending the instructions to a lab with a model of the patient's teeth (e.g., a dental impression/cast or digital model) for fabrication. Once received, the patient information is entered into a database and the case may be scheduled for the design review. A designer may review the prescription and make any needed modifications for the technicians, who will be making the appliance and in response to the prescription.

This process may be time consuming and only provides the dental professional (e.g., dentist, orthodontist, etc.) with a limited amount of control and input in designing the appliance for the patient. In particular, the dental professional is not easily permitted to iterate on the design, as the cost of submitting changes to the design may be high. Described herein are methods and apparatuses that may address these needs.

### SUMMARY OF THE DISCLOSURE

Described herein are dynamic prescription interfaces, including systems and apparatuses assisting a dental professional (e.g., dentist) in prescribing one or more dental appliances (e.g., retainers, expanders, aligners, etc.). Also described herein are methods and apparatuses for fabricating one or more dental appliances using these dynamic prescription interfaces.

The methods and apparatuses (devices, systems, etc., including software, hardware and/or firmware) described herein may visually show appliance design updates in real-time or near real-time on a user interface of a prescription form. The appliance(s) may be initially generated from pre-set preferences that may be used to initially and automatically fill out most of the appliance device information of the prescription and may be sequentially modified as the user modifies and/or inputs the prescription. For example, the appliance design preview on the user interface for the prescription form may be age-selected between child, teen and adult; in some cases the child product type may include a retainer appliance design that highlights mixed dentition and solutions for unerupted teeth.

In some examples, the prescription user interface described herein may pre-populate the fields of the prescription and may generate an image of one or more (e.g., an upper and a lower) dental appliance based on the current prescription information as the dental professional enters or modifies the various fields of the prescription. As mentioned, the user interface may modify the image of the one or more dental appliances based on the age of the patient entered by the dental professional (e.g., for kids, teens, and adults). In general, these methods may allow real-time or near real-time viewing of appliance design features. Pre-set templates, with features already selected in prescription input fields (e.g., the prescription form or Rx-form) may be based on prescription parameters that are either the most commonly assigned fields based on population data (across multiple different dental professionals) or specific to a particular dental professional, e.g., such as selected data input that doctors would frequently prescribe. For example, if the prescription is for a retainer, the templates provided may have common retainer features pre-set at the arch level and can be customizable for the dental professional to create their own template from scratch or alter one provided by the apparatus or method, and may save any changes for future prescription (Rx) submissions.

In some cases, unique features like eruption compensation may be visualized on an appliance preview with unique visualizations at the tooth-level. Also, the system and apparatus may help the doctor with tooth charting, e.g., by prefilled tooth charts according to the patient's age, as mentioned above. This may provide models of the dentition that may be configured to match the approximate age group (i.e. children, teens, adults) of the intended patient.

For example, described herein are non-transitory computing device readable media having instructions stored thereon that are executable by a processor to cause a computing device to: receive or access patient information including a patient's age data; pre-populate one or more prescription fields of a patient prescription based on the patient information; select a representative dental appliance based on the one or more prescription fields for the patient's upper and/or lower dental arch; display the representative dental appliance for the upper and/or lower dental arch in a user interface; iteratively receive user modification to one or more prescription fields from the user interface and modify the displayed representative dental appliance for the upper and/or lower dental arch according to the modified prescription including the modified prescription fields; and output a final patient prescription based on the modified prescription field following a user input indicating acceptance of the modified representative dental appliance. Any of these apparatuses may be configured to manufacture (e.g., to fabricate or cause to be fabricated) one or more dental appliances based on the final patient prescription.

The instructions may be further configured to cause the computing device to receive or access dental practitioner information. In some cases, the instructions may be configured to cause the computing device to receive, access or generate a three-dimensional (3D) digital model of the patient's upper and/or lower arch. For example, the 3D digital model of the patient's upper and/or lower arch may be based on scan data. The instructions may be further configured to cause the computing device to pre-populate the one or more prescription fields of the patient prescription based on the patient's age. The instructions may be further configured to cause the computing device to modify the displayed representative dental appliance in real time. The instructions may be configured to display one or more prescription fields in the user interface and to allow the user to change the one or more fields. The instructions may be configured to include to rotate, in three-dimensional space, the displayed representative dental appliance for the upper and/or lower dental arch. The instructions may be configured to display the representative dental appliance for the upper and/or lower dental arch in the user interface in combination with a digital model of the patient's upper and/or lower dental arch.

For example, a non-transitory computing device readable medium may have instructions stored thereon that are executable by a processor to cause a computing device to: receive or access patient information including a patient's age data; receive or access a treatment type; pre-populate one or more prescription fields of a patient prescription based on the patient information; select a representative dental appliance based for the patient's upper and/or lower dental arch based on the patient's age and the treatment type; display the representative dental appliance for the upper and/or lower dental arch in a user interface; iteratively receive user modification to one or more prescription fields from the user interface and modify the displayed representative dental appliance for the upper and/or lower dental arch according to the modified prescription including the modified prescription fields; and output a final patient prescription based on the modified prescription field following a user input indicating acceptance of the modified representative dental appliance.

Also described herein are methods comprising: receiving or accessing patient information including a patient's age data; pre-populating one or more prescription fields of a patient prescription based on the patient information; selecting a representative dental appliance based on the one or more prescription fields for the patient's upper and/or lower dental arch; displaying the representative dental appliance for the upper and/or lower dental arch in a user interface; iteratively receiving user modification to one or more prescription fields from the user interface and modify the displayed representative dental appliance for the upper and/or lower dental arch according to the modified prescription including the modified prescription fields; and outputting a final patient prescription based on the modified prescription field following a user input indicating acceptance of the modified representative dental appliance. Any of these methods may include fabricating one or more dental appliances based on the final patient prescription. The method may include accessing dental practitioner information. The methods may include receiving, accessing or generating a three-dimensional (3D) digital model of the patient's upper and/or lower arch. The 3D digital model of the patient's upper and/or lower arch may be based on scan data. The method may include pre-populating the one or more prescription fields of the patient prescription based on the patient's age.

As used herein, fabricating one or more dental appliances based on the final patient prescription may include fabricating by an automated or semi-automated method, including 3D printing. The step of fabricating may include sending instructions (e.g., including an electronic file) with instructions to form one or more dental appliances based on the final patient prescription.

Any of these methods may include displayed representative dental appliance in real time. The methods may include having one or more prescription fields in the user interface and to allow the user to change the one or more fields. The method may include rotating, in three-dimensional space, the displayed representative dental appliance for the upper and/or lower dental arch. Any of these methods may include displaying the representative dental appliance for the upper and/or lower dental arch in the user interface in combination with a digital model of the patient's upper and/or lower dental arch.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 schematically illustrates one example of a method as described herein.

FIG. 2A is one example of a user interface as described herein.

FIG. 2B is an example of a user interface as described herein.

FIG. 3 shows a diagram illustrating an example of a computing environment including the dynamic prescription interface module(s), described herein.

FIGS. 4A-4K illustrate examples of a user interface, including multiple screens, for one exemplary embodiment.

### DETAILED DESCRIPTION

The dynamic prescription interfaces described herein improve the speed and efficiency of the dental and/or orthodontic prescription process, at least in part by reducing the number of button clicks and time that a dental practitioner would need to perform to submit a prescription form. These methods and apparatuses (e.g., devices, systems, etc.) may use pre-set templates. However, such templates may be dynamically selected based on just a few factors, such as patient age and dental practitioner region and/or identity. Moreover the template may be modified by the user. For example a dental practitioner (e.g., doctor, orthodontist, dentist, etc.) may use a template, update a template, or create their template of pre-filled preferences for features. In some cases, the methods and apparatuses described herein may be configured to present a visualization (e.g., image) of the appliance design with unique features at the tooth-level customization. The appliance may be configured to rotate (or be rotated) in 3D spatial dimensions as part of the design preview, as certain features may best be viewed by different angles. The system may be configured to permit visualization of both arches in the appliance design preview. In some cases, having the prescription, an intraoral scan, and an appliance design visualization in one view may help ensure accuracy and not having to refer to charts when filling out the prescription form. These methods and apparatuses may also reduce the number of clicks needed when tooth charting because tooth charts reflect typical dentition for a patient and reflect the age of the patient. Thus, in any of these examples, an anatomy may be pre-loaded with a common dentition used for each age group (e.g., child, young adult, and adult). The methods and apparatuses may be configured to use a pre-filled preference feature. In some cases the pre-filled preferences may be different between the upper and lower arch. This may further reduce the time needed to fill out the prescription form for each arch.

These methods and apparatuses may enable the dental practitioner to design an appliance, in some examples a retainer, by themselves and see the real-time or nearly real-time updates. As the dental practitioner (e.g., doctor or doctor's staff), confirms the dental anatomy, and selects either a defined preset or their own defined retainer template, the live update showing appliance design overlayed on top of the patient's arch will be available as the dental practitioner continues to refine, customize, add or remove individual features. This can help the dental practitioner understand, in real time, if a certain feature is suitable or not for the patient, and may aid in visualizing the final design of the appliance and may allow rotation of the appliance in 3D viewer as well. This may enhance the doctor's experience while ordering the retainer appliance for this patient. These methods and apparatuses may be used with any number of treatment modalities and appliances (e.g., retainers, expanders, aligners, etc.).

These methods and apparatuses may be configured to generate the final prescription information and to confirm (e.g., visually, as part of the user interface) the prescription features, including the type of dental appliance, the number of dental appliances, and features of the dental appliance. Once the prescription information is complete, the apparatus and methods may pass it on to one or more sub-systems to generate the treatment plan and/or fabrication, including generating a file (e.g., digital file) with the instructions for forming the dental appliances.

In some cases the methods and apparatuses may be configured to be performed directly on a scanner (e.g., intraoral scanner). In some cases, these methods and apparatuses may be configured to be performed on a mobile platform (e.g., smartphone, tablet, etc.).

Any of these methods and apparatuses may be configured to provide a series of dental appliances that may each have different ship dates and/or features for each set of retainers. In some cases the methods or apparatuses may be configured to provide multiple sets of retainers and each set may have different features.

In general, any of these methods and apparatuses may receive one or more scan of the patient's dentition. In some cases the methods or apparatus may use a single scan; e.g., the order (prescription) may be based upon a single scan. In some cases the prescription may include placement of attachments (e.g., hooks and/or button features on the teeth and/or appliance). In some cases the dental appliance(s) may be configured to maintain class II correction; the attachments may be removed in other sets of retainers or may be used by other sets of retainers.

In some cases the prescription (e.g., order) can include multiple sets of retainers and an individual subset of the retainer order may be sent at a different time. The method and apparatus may be configured to be updated based upon a new & different scan. This may allow the use of earlier prescription information with later scans, for example, if a growing child has a first set order (e.g., including four sets of dental appliances) and scans are taken after one or more set is ordered; an additional scan may be taken after a period of time, later once the patient is older, and this new scan (rescan) may be used to generate another set of retainers and may be shipped based upon the new scan; the same prescription or an updated prescription may be used. In some cases the system or apparatus may receive the prior prescription as the preset values, but may allow the dental practitioner to adjust them as needed.

In some cases, the preset values provided to the method or apparatus may be based on age, as described above. For example preset values for these method and apparatuses may select from a library of preset values (e.g., treatment parameters, dental appliance examples/types, etc.) based on the patient current age within certain categories (E.g., child, teen/young adult, and adult). In some cases teens and adults may have preset values and dentition models and/or dental appliance models that are configured specifically for teens and/or adults, and separate preset values, dentition models and/or appliance models that are configured for use with children. Thus, in general, these methods and apparatuses may use different preset templates per age along with different pre-set tooth charting defaults based on age.

In general, these methods and apparatuses may include auto-segmentation of scan data from the patient. These methods and apparatuses may not require tooth charting.

The methods and apparatuses may be configured to the design of the selected type of appliance (e.g., retainer, expander, aligner, etc.) according to the arch type, size and dental anatomy of the patient. This information may be received as a digital scan of the patient's teeth. In some cases, the methods and apparatuses described herein may allow a dental practitioner to use and/or order a particular stage of treatment of a dental treatment. For example, these methods and apparatuses may permit the user to use a past treatment stage (e.g., a scan or digital model based on a prior scan or derived from a prior scan) that may be used instead of a new scan to leverage the existing treatment stage.

In any of these methods and apparatuses, prior treatment information specific to the patient may be imported and used when generating the prescription form for a new treatment. For example, these methods and apparatuses may integrate preference or features from a prior treatment (such as a treatment to align the teeth) as part of a later treatment, such as a new alignment treatment and/or a retainer treatment.

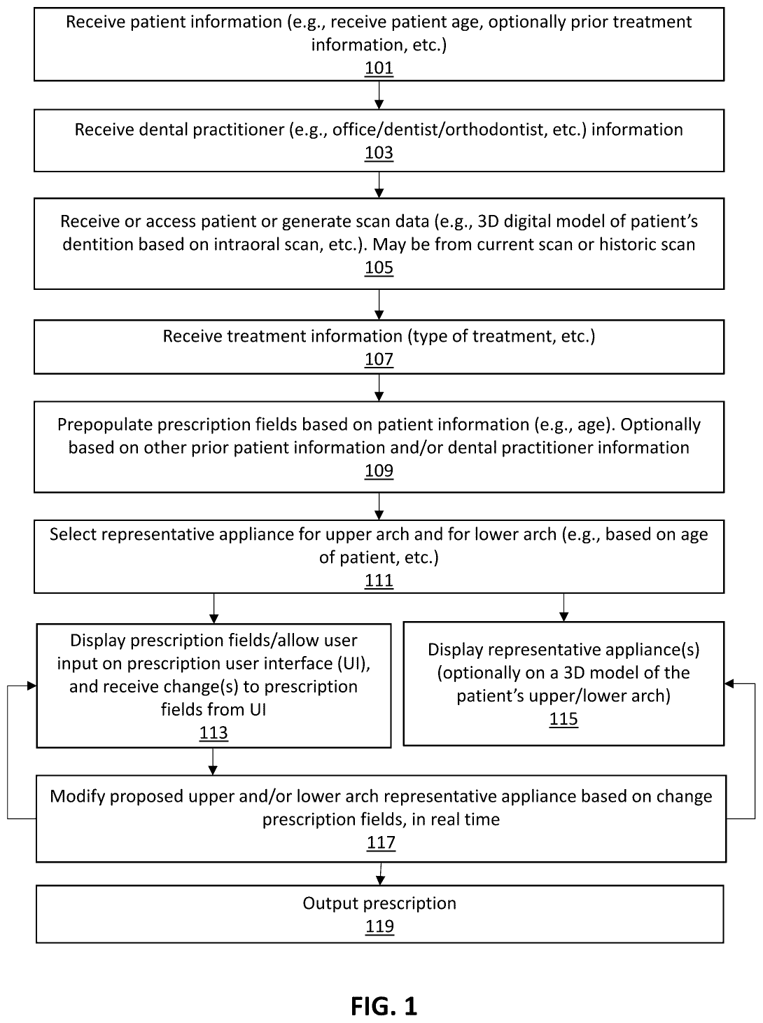

FIG. 1 illustrates one example of an overview of the methods (and operation of an apparatus) for dynamically taking a prescription as described herein. As shown in FIG. 1 the method may begin by receiving input, e.g., from a user, such as a dental practitioner, indicating a new prescription (or accessing an existing prescription) for a particular patient 101. The user may access this information from a user interface and may input or provide access to patient information, such as patient age, prior treatment(s), etc. The user may optionally be linked or identified with a particular dental practitioner and/or dental and/or orthodontic practice 103. The method may then receive and/or access and/or generate a three-dimensional (3D) model of the patient's dental arch(s) 105. The user may also input, e.g., through the user interface, the type of dental appliance being prescribed 107, such as one or more retainers, expanders, etc.

The initial input may be handled by a patient data input module, which may access a user interface. The patient data input module may be part of the overall dynamic prescription engine or module. The patient data input module may access one or more databases, including patient information databases, and/or patient scan/digital model databases, etc. The 3D digital model of the patient's dental arch (e.g., upper and/or lower arch) may be processed by one or more additional sub-systems or modules and the 3D digital model may be segmented so as to include individual teeth.

These methods and apparatuses may also include or be configured to include one or more prescription pre-population modules that may receive data from the patient data input module (e.g., patient age, type of treatment, etc.) and may identify a template based on this information from one or more databases of prescription templates to prepopulate the prescription fields of the user interface 109. In some cases, as described above, the template may be based at least in part on the age of the patient. For example, the template may be specific to a child, young adult/teen, and adult.

The user interface may be displayed including the pre-populated prescription fields 113 and, concurrently, one or more displays of the proposed dental appliance; the dental appliance may be shown on the upper arch/lower arch model of the patient's teeth. The upper arch/lower arch model(s) may be derived from the scan of the patient's actual dentition. Optionally in some cases the displayed upper and/or lower dentition may instead by based on a model of the upper and/or lower dentition that is not specific to the patient but may be from (and/or derived from) a generic dentition model; in some cases this generic dentition model may be modified, e.g., in pre-determined manner, to better reflect the patient's dentition (e.g., removing teeth, scaling, etc.). In some cases the upper arch/lower arch dentition model(s) may be selected from a library of upper arch/lower arch models to best match the patient's age and/or dentition. The user may adjust the display, e.g., to rotate the display, and/or the display may be automatically rotated/adjusted 115. As the user modified (e.g. selects, as from a pull-down menu, or otherwise inputs) the prescription fields in the user interface, the display of the proposed representative appliance(s) may be adjusted 117, e.g., in real-or near-real time. This process may be continuous and iterated. The user may determine when the process is complete, and may output the prescription, electronically or manually, for fabrication of the dental appliance(s) 119.

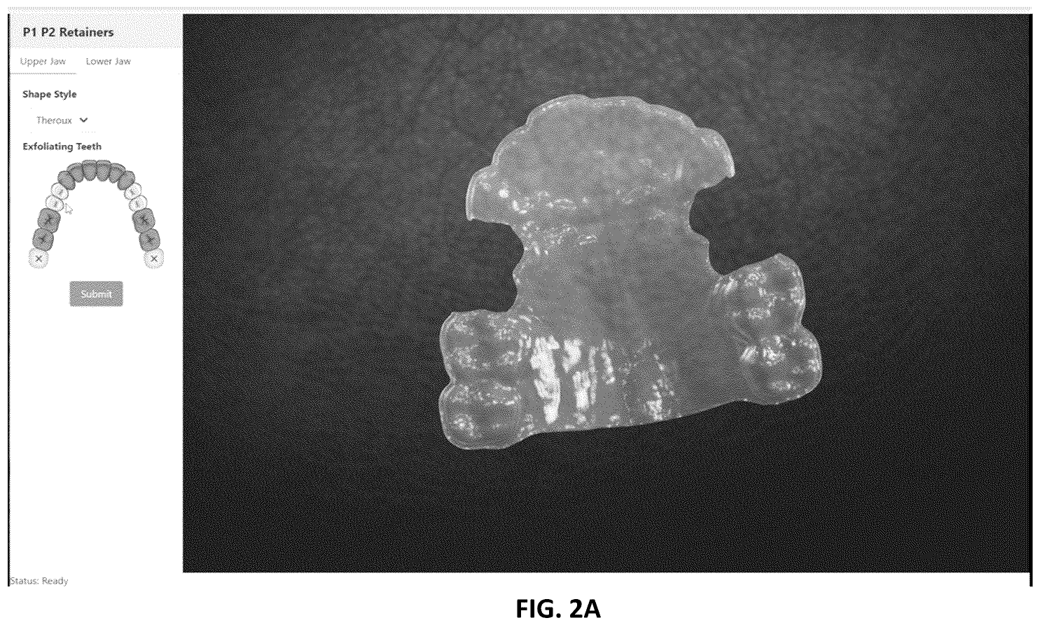

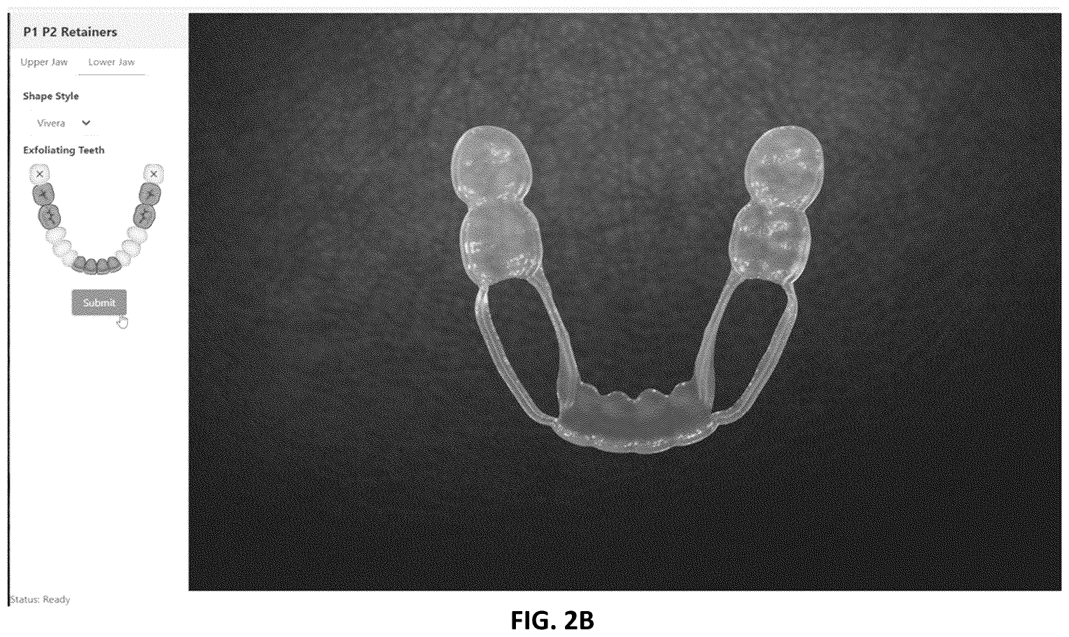

In general, these methods may include a user interface that includes both a prescription interface portion and a display portion, e.g., displaying one or both upper and lower arches representing the patient's dental arch and showing a mock-up of a dental appliance that may be dynamically modified, e.g., in real-or near-real time as the user modifies the prescription entry portion of the user interface. For example, FIGS. 2A-2B illustrate examples of portions of a user interface. As described above, these methods and apparatuses may display a visual representation of the customization choices to the user (e.g., dental practitioner), with real time updates. The graphics may be pre-rendered, or work with a generic dentition model, to avoid the need for real-time processing of patient scan.

FIG. 2A shows a first example of a portion of a user interface as described herein. In FIG. 2A the left side of the user interface shows prescription information, including the type of appliance (e.g. retainer) and the shape style of the retainer (e.g., “Theroux”) and allows toggling between the upper and lower jaw. The upper jaw is shown. The prescription including a line model of the teeth, indicating exfoliating teeth. The user input also includes a “submit” button. The right side of the example shown in FIG. 2A illustrates an example rendering of the appliance, based on the prescription information, in this case, showing a Theroux-type retainer for the upper teeth.

FIG. 2B shows another example of the user interface, showing the lower jaw. The user interface portion on the right allows toggling between the upper and lower jaw, and selection of the type of retainer (e.g., “Vivera”) as well as showing a line model of the lower arch, indicating exfoliating teeth. The right side of the portion of the user interface shown in FIG. 2B shows a 3D rendering of the lower law retainer.

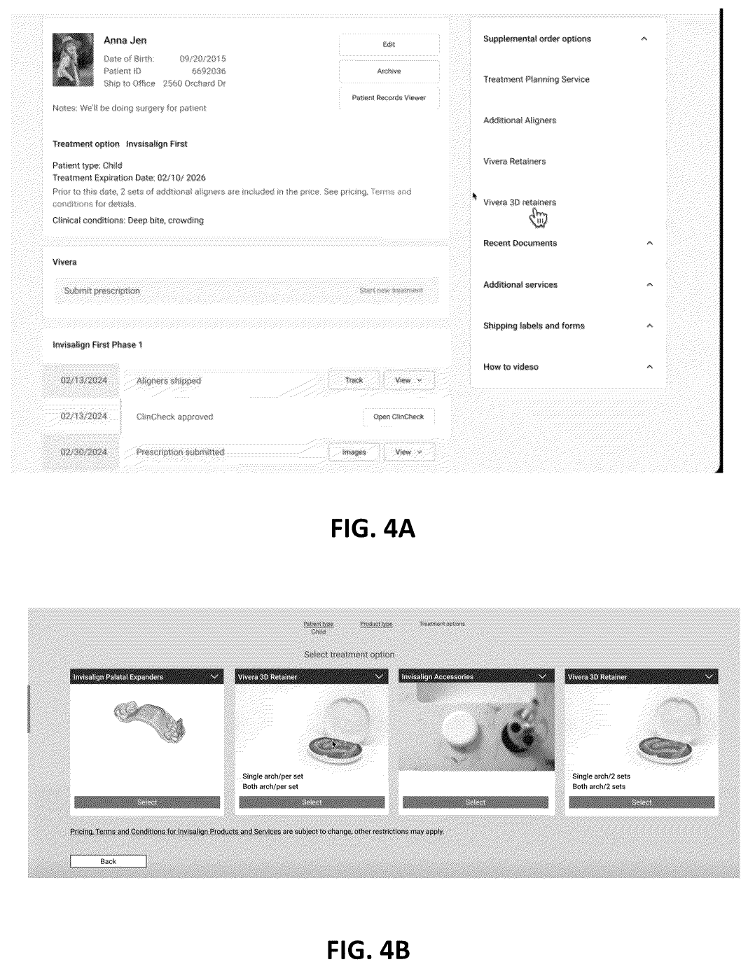

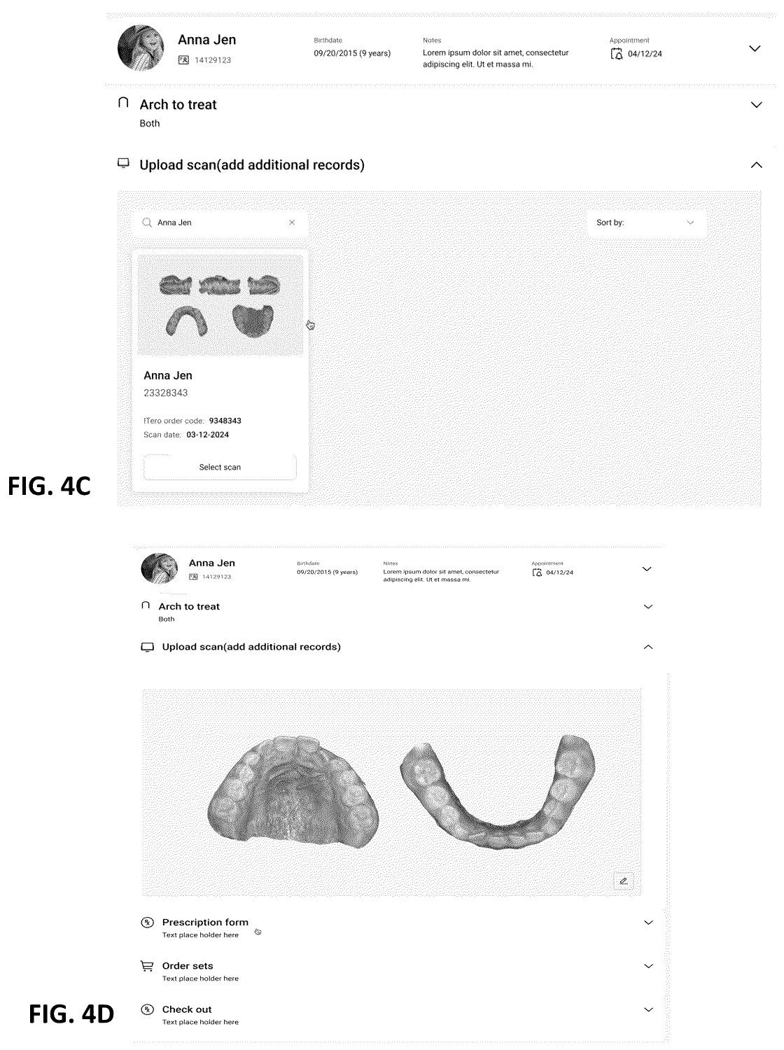

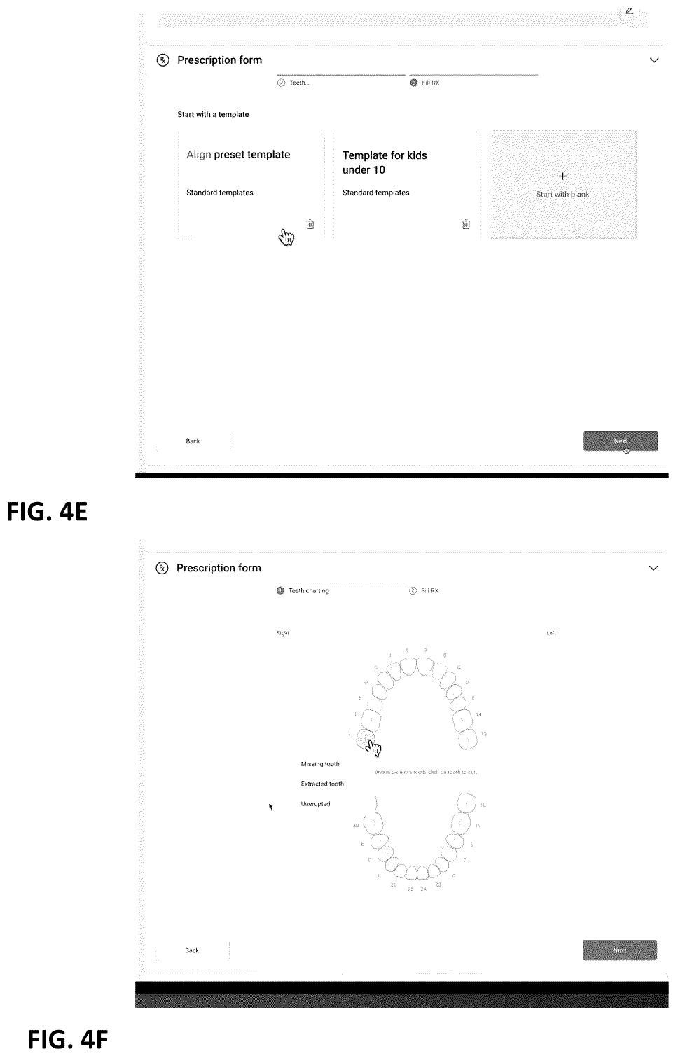

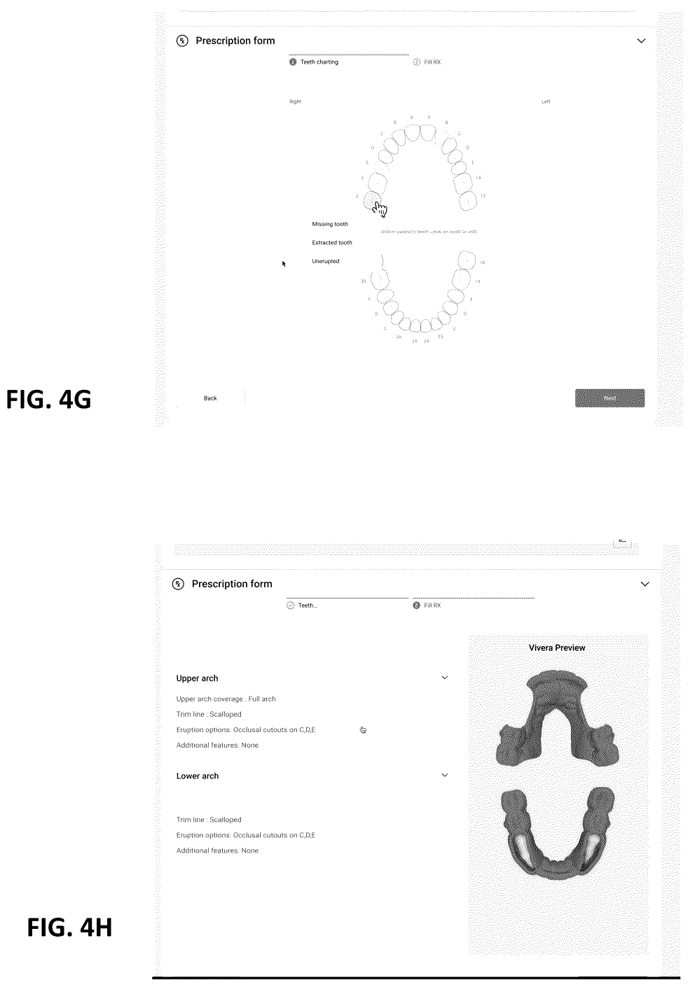

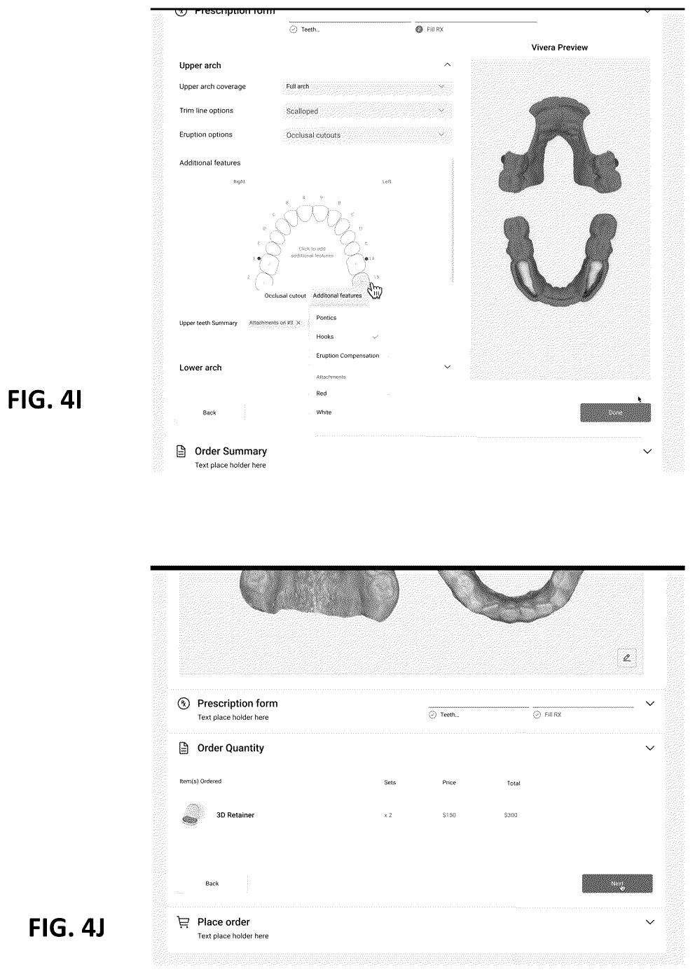

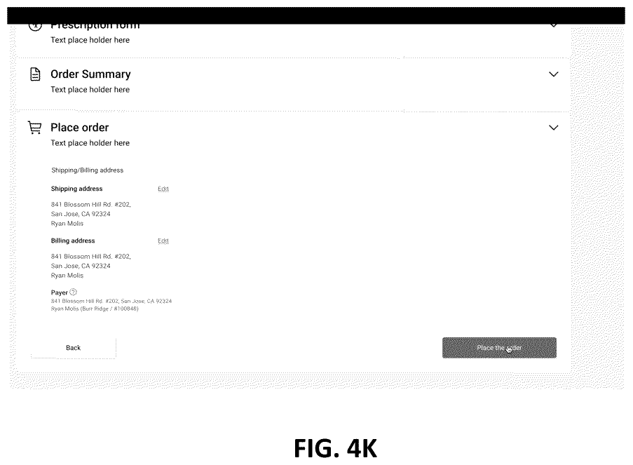

FIGS. 4A-4K illustrate another example of a user interface. FIGS. 4A-4D illustrate one, non-limiting, example of a method and interface for entering patient information, and selecting a dental product. FIGS. 4E-4I illustrate one example of a dynamic prescription user interface as described herein. FIGS. 4J-4K show examples of finalizing and ordering the dental appliance as described.

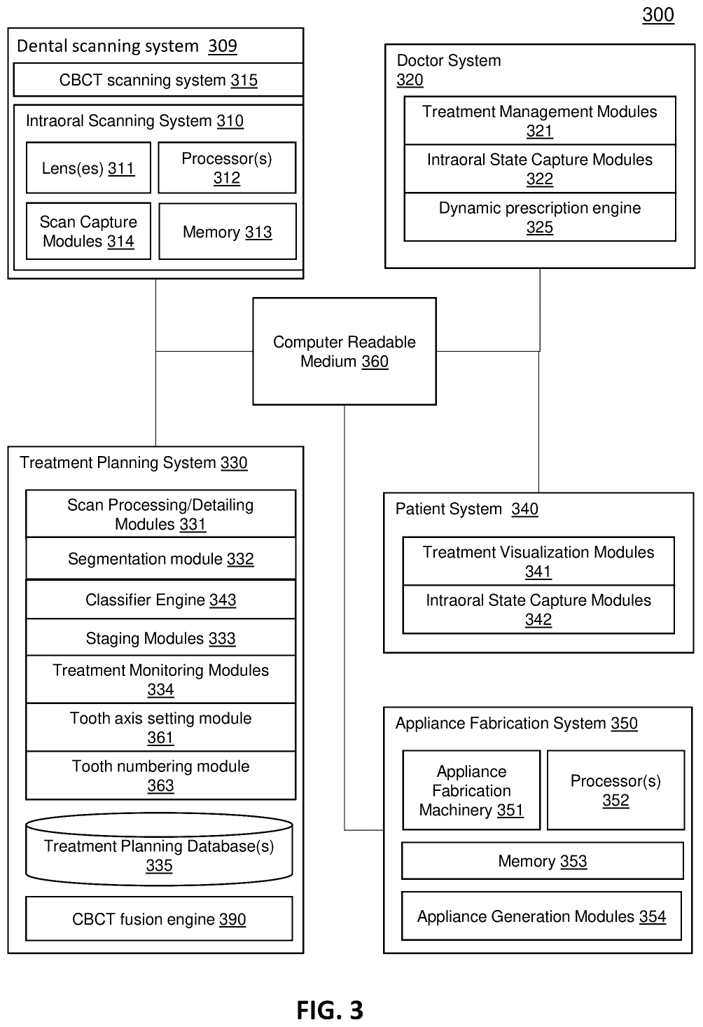

FIG. 3 is a diagram illustrating one variation of a computing environment 300 that may include and/or implement the methods for dynamic prescription as described herein, under the direction of a dental professional. The computing environment may include a dynamic prescription engine or module, configured to operate as just described. The example computing environment 300 shown in FIG. 3 includes a dental scanning system (e.g., optionally including an intraoral scanning system 310, and/or CBCT scanning system 315), a doctor system 320, which is shown including the dynamic prescription engine 325, a treatment planning system 330 (e.g., technician system), a patient system 340, an appliance fabrication system 350, and computer-readable medium 30. Each of these systems may be referred to equivalently as a sub-system of the overall system (e.g., computing environment). Although shown as discrete systems, some or all of these systems may be integrated and/or combined. In some variations a computing environment (dental computing system) 300 may include just one or a subset of these systems (which may also be referred to as sub-systems of the overall system 300). As mentioned, one or more of these systems may be combined or integrated with one or more of the other systems (sub-systems), such as, e.g., the patient system and the doctor system may be part of a remote server accessible by doctor and/or patient interfaces. The computer readable medium 30 may be divided between all or some of the systems (subsystems); for example, the treatment planning system and appliance fabrication system may be part of the same sub-system and may be on a computer readable medium 30. Further, each of these systems may be further divided into sub-systems or components that may be physically distributed (e.g., between local and remote processors, etc.) or may be integrated. Although the dynamic prescription engine 325 is shown as part of the doctor system 320, it may be part of and/or may access other systems/sub-systems, such as the dental scanning system 309, treatment planning system 320, appliance fabrication system 350, etc.

A dental scanning system may include one or more of: an intraoral scanning system 310, and/or CBCT scanning system 315. The intraoral scanning system may include an intraoral scanner as well as one or more processors for processing images. For example, an intraoral scanning system 310 can include optics 311 (e.g., one or more lenses, filters, mirrors, etc.), processor(s) 33, a memory 313, and a scan capture module 314. In general, the intraoral scanning system 310 can capture one or more images of a patient's dentition. Use of the intraoral scanning system 310 may be in a clinical setting (doctor's office or the like) or in a patient-selected setting (the patient's home, for example). In some cases, operations of the intraoral scanning system 310 may be performed by an intraoral scanner, dental camera, cell phone or any other feasible device.

The optical components 311 may include one or more lenses and optical sensors to capture reflected light, particularly from a patient's dentition. The scan capture module 314 can include instructions (such as non-transitory computer-readable instructions) that may be stored in the memory 313 and executed by the processor(s) 312 to can control the capture of any number of images of the patient's dentition.

In FIG. 3 the segmentation 332 and verification 332 (e.g., classifier engine 343) are shown as part of the treatment planning sub-system 330, however in some examples some, or all, of these components may be part of (or duplicated in) the dental scanning system 309. For example, the segmentation module 332 may be in the intraoral scanning sub-system 310 or another sub-system may be in the treatment planning sub-system 330 or some other sub-system. Any of the component systems or sub-systems of the dental computing environment 300 may access or use the segmented data. Similarly the tooth axis setting module 361 and/or the tooth numbering module 363 may be part of the treatment planning sub-system 330, however in some examples some, or all, of these components may be part of (or duplicated in) the dental scanning system 309.

The doctor system 320 (e.g., doctor sub-system) may include the dynamic prescription engine 325. In some cases, the doctor system may include a treatment management module 321 and an intraoral state capture module 322 that may access or use the 3D model based on segmented data. The doctor system 320 may provide a “doctor facing” interface to the computing environment 300. The treatment management module 321 can perform any operations that enable a doctor or other clinician to manage the treatment of any patient. In some examples, the treatment management module 321 may provide a visualization and/or simulation of the patient's dentition with respect to a treatment plan. This user interface may also include display the segmentation. Thus, the doctor system may include the user interface.

The intraoral state capture module 322 can provide images of the patient's dentition to a clinician through the doctor system 320. The images may be captured through the dental scanning system 309 and may also include images of a simulation of tooth movement based on a treatment plan.

In some examples, the treatment management module 321 can enable the doctor to modify or revise a prescription and/or treatment plan, particularly when images provided by the intraoral state capture module 322 indicate that the movement of the patient's teeth may not be according to the treatment plan. The doctor system 320 may include one or more processors configured to execute any feasible non-transitory computer-readable instructions to perform any feasible operations described herein.

Alternatively or additionally, the treatment planning system 330 may include any of the methods and apparatuses described herein, including the CBCT fusion engine. Although the CBCT fusion engine is shown as part of the treatment planning system 330, the CBCT fusion engine may be separate and may communicate directly or indirectly with the Doctor System 320 and/or the treatment planning system 330, or any other system or sub-system shown.

The treatment planning system 330 may include scan processing/detailing module 331, segmentation module 332, classifier engine(s) 343, staging module 333, and treatment monitoring module 334, and a treatment planning database(s) 335. In general, the treatment planning system 330 can determine a treatment plan for any feasible patient. The scan processing/detailing module 331 can receive or obtain dental scans (such as scans from the dental scanning system 309) and can process the scans to “clean” them by removing scan errors and, in some cases, enhancing details of the scanned image. The treatment planning system 330 may perform segmentation. For example, a treatment planning system may include a segmentation module 332 that can segment a dental model into separate parts including separate teeth, gums, jaw bones, and the like. In some cases, the dental models may be based on scan data from the scan processing/detailing module 331 (and/or classifier engine 343).

The staging module 333 may determine different stages of a treatment plan. Each stage may correspond to a different dental aligner. The staging module 333 may also determine the final position of the patient's teeth, in accordance with a treatment plan. Thus, the staging module 333 can determine some or all of a patient's orthodontic treatment plan. In some examples, the staging module 333 can simulate movement of a patient's teeth in accordance with the different stages of the patient's treatment plan.

An optional treatment monitoring module 334 can monitor the progress of an orthodontic treatment plan. In some examples, the treatment monitoring module 334 can provide an analysis of progress of treatment plans to a clinician. Although not shown here, the treatment planning system 330 can include one or more processors configured to execute any feasible non-transitory computer-readable instructions to perform any feasible operations described herein.

The Tooth axis setting module 361 and/or Tooth numbering module 363 may be included as part of the treatment planning sub-system 330 as discussed above, and may include the features and perform the steps described above for either tooth numbering/setting or verification and/or for tooth axis determination, setting and/or verification.

The patient system 340 can include a treatment visualization module 341 and an intraoral state capture module 342. In general, the patient system 340 can provide a “patient facing” interface to the computing environment 300. The treatment visualization module 341 can enable the patient to visualize how an orthodontic treatment plan has progressed and also visualize a predicted outcome (e.g., a final position of teeth).

In some examples, the patient system 340 can capture dentition scans for the treatment visualization module 341 through the intraoral state capture module 342. The intraoral state capture module can enable a patient to capture his or her own dentition through the dental scanning system 309. Although not shown here, the patient system 340 can include one or more processors configured to execute any feasible non-transitory computer-readable instructions to perform any feasible operations described herein.

The appliance fabrication system 350 can include appliance fabrication machinery 351, processor(s) 352, memory 353, and appliance generation module 354. In general, the appliance fabrication system 350 can directly or indirectly fabricate aligners to implement an orthodontic treatment plan. In some examples, the orthodontic treatment plan may be stored in the treatment planning database(s) 335. Any of these apparatuses and methods may be configured to include the step of fabricating one or more (e.g., a series) of dental appliances using a 3D model (e.g., including using the corrected segmentation, as described herein).

The appliance fabrication machinery 351 may include any feasible implement or apparatus that can fabricate any suitable dental aligner. The appliance generation module 354 may include any non-transitory computer-readable instructions that, when executed by the processor(s) 352, can direct the appliance fabrication machinery 351 to produce one or more dental aligners. The memory 353 may store data or instructions for use by the processor(s) 352. In some examples, the memory 353 may temporarily store a treatment plan, dental models, or intraoral scans.

The computer-readable medium 30 may include some or all of the elements described herein with respect to the computing environment 300. The computer-readable medium 30 may include non-transitory computer-readable instructions that, when executed by a processor, can provide the functionality of any device, machine, or module described herein.

As mentioned, the systems and sub-systems described herein, including the dynamic prescription engine, may include a trained pattern matching agent may an artificial intelligence agent, including a machine learning agent. The machine learning agent may be a deep learning agent. In some examples, the trained pattern matching agent may be trained neural network. Any appropriate type of neural network may be used, including generative neural networks. The neural network may be one or more of: perceptron, feed forward neural network, multilayer perceptron, convolutional neural network, radial basis functional neural network, recurrent neural network, long short-term memory (LSTM), sequence to sequence model, modular neural network, etc. In some examples a trained pattern matching agent may be trained using a training data set.

The apparatuses described herein may include on or more processors. A processor may include hardware that runs computer program code configured to perform these methods. The term ‘processor’ may include a controller and may encompass not only computers having different architectures such as single/multi-processor architectures and sequential (Von Neumann)/parallel architectures but also specialized circuits such as field-programmable gate arrays (FPGA), application specific circuits (ASIC), signal processing devices and other devices.

The dynamic prescription engine/modules may comprise one or more engines and datastores. A computer system can be implemented as an engine, as part of an engine or through multiple engines. As used herein, an engine may include one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. Depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method, the method comprising:
receiving or accessing patient information including a patient's age data;
pre-populating one or more prescription fields of a patient prescription based on the patient information;
selecting a representative dental appliance based on the one or more prescription fields for the patient's upper and/or lower dental arch;
displaying the representative dental appliance for the upper and/or lower dental arch in a user interface;
iteratively receiving user modification to one or more prescription fields from the user interface and modify the displayed representative dental appliance for the upper and/or lower dental arch according to the modified prescription including the modified prescription fields;
outputting a final patient prescription based on the modified prescription field following a user input indicating acceptance of the modified representative dental appliance;
fabricating one or more dental appliances based on the final patient prescription.

2. The method of claim 1, further comprising accessing dental practitioner information.

3. The method of claim 1, further comprising receiving, accessing or generating a three-dimensional (3D) digital model of the patient's upper and/or lower arch.

4. The method of claim 3, wherein the 3D digital model of the patient's upper and/or lower arch is based on scan data.

5. The method of claim 1, further comprising pre-populating the one or more prescription fields of the patient prescription based on the patient's age.

6. The method of claim 1, modifying the displayed representative dental appliance in real time.

7. The method of claim 1, displaying one or more prescription fields in the user interface and to allow the user to change the one or more fields.

8. The method of claim 1, rotating, in three-dimensional space, the displayed representative dental appliance for the upper and/or lower dental arch.

9. The method of claim 1, displaying the representative dental appliance for the upper and/or lower dental arch in the user interface in combination with a digital model of the patient's upper and/or lower dental arch.

10. A non-transitory computing device readable medium having instructions stored thereon that are executable by a processor to cause a computing device to:
receive or access patient information including a patient's age data;
pre-populate one or more prescription fields of a patient prescription based on the patient information;
select a representative dental appliance based on the one or more prescription fields for the patient's upper and/or lower dental arch;
display the representative dental appliance for the upper and/or lower dental arch in a user interface;
iteratively receive user modification to one or more prescription fields from the user interface and modify the displayed representative dental appliance for the upper and/or lower dental arch according to the modified prescription including the modified prescription fields;
output a final patient prescription based on the modified prescription field following a user input indicating acceptance of the modified representative dental appliance; and
manufacture one or more dental appliances based on the final patient prescription.

11. The non-transitory computing device readable medium of claim 10, wherein the instructions are further configured to cause the computing device to receive or access dental practitioner information.

12. The non-transitory computing device readable medium of claim 10, wherein the instructions are further configured to cause the computing device to receive, access or generate a three-dimensional (3D) digital model of the patient's upper and/or lower arch.

13. The non-transitory computing device readable medium of claim 12, wherein the 3D digital model of the patient's upper and/or lower arch is based on scan data.

14. The non-transitory computing device readable medium of claim 10, wherein the instructions are further configured to cause the computing device to pre-populate the one or more prescription fields of the patient prescription based on the patient's age.

15. The non-transitory computing device readable medium of claim 10, wherein the instructions are further configured to cause the computing device to modify the displayed representative dental appliance in real time.

16. The non-transitory computing device readable medium of claim 10, wherein the instructions are further configured to display one or more prescription fields in the user interface and to allow the user to change the one or more fields.

17. The non-transitory computing device readable medium of claim 10, wherein the instructions are further configured to include to rotate, in three-dimensional space, the displayed representative dental appliance for the upper and/or lower dental arch.

18. The non-transitory computing device readable medium of claim 10, wherein the instructions are further configured to display the representative dental appliance for the upper and/or lower dental arch in the user interface in combination with a digital model of the patient's upper and/or lower dental arch.

19. A non-transitory computing device readable medium having instructions stored thereon that are executable by a processor to cause a computing device to:
receive or access patient information including a patient's age data;
receive or access a treatment type;
pre-populate one or more prescription fields of a patient prescription based on the patient information;
select a representative dental appliance for the patient's upper and/or lower dental arch based on the patient's age and the treatment type;
display the representative dental appliance for the upper and/or lower dental arch in a user interface;
iteratively receive user modification to one or more prescription fields from the user interface and modify the displayed representative dental appliance for the upper and/or lower dental arch according to the modified prescription including the modified prescription fields; and
output a final patient prescription based on the modified prescription field following a user input indicating acceptance of the modified representative dental appliance.
